(19) United States
(12) Patent Application Publication
(10) Pub. No.: US 2018/0250522 A1
(43) Pub. Date: Sep. 6, 2018

(54) TRANSCEIVER OF GENERATING MICROWAVE SIGNAL AND THERAPY SYSTEM INCLUDING THE TRANSCEIVER

(71) Applicant: Electronics and Telecommunications Research Institute, Daejeon (KR)

(72) Inventors: Soon Ik JEON, Daejeon (KR); Jang Yeol KIM, Daejeon (KR); Bo Ra KIM, Daejeon (KR); Seong-Ho SON, Daejeon (KR); Kwang Jae LEE, Daejeon (KR)

(73) Assignee: Electronics and Telecommunications Research Institute, Daejeon (KR)

(21) Appl. No.: 15/614,392

(22) Filed: Jun. 5, 2017

(30) Foreign Application Priority Data
Mar. 6, 2017 (KR) 10-2017-0028276

(51) Int. Cl. A61N 5/02; A61N 1/39; A61B 17/22; A61B 18/18

(52) U.S. Cl. CPC A61N 5/02; A61N 1/395; A61B 2090/3782; A61B 18/1815; A61B 17/2202

(57) ABSTRACT

Disclosed is a transceiver of outputting a microwave signal and a therapy system including the transceiver, the transceiver including a signal attenuator to control an intensity of a microwave signal based on corrected information, a phase shifter to control a phase of the microwave signal based on the corrected information, a signal switch to control a turning-on/off of the microwave signal based on on/off information, a power amplifier to amplify the microwave signal, a detector to calculate a difference between the microwave signal and a result of the amplifying, and a self-monitoring controller to generate the corrected information based on the difference, phase information, and intensity information, output the corrected information to the signal attenuator and the phase shifter, generate the on/off information, and output the on/off information to the signal switch.

### CROSS-REFERENCE TO RELATED APPLICATION(S)

This application claims the priority benefit of Korean Patent Application No. 10-2017-0028276 filed on Mar. 6, 2017 in the Korean Intellectual Property Office, the disclosure of which is incorporated herein by reference for all purposes.

### BACKGROUND

### 1. Field

One or more example embodiments relate to a transceiver of generating a microwave signal and a therapy system including the transceiver.

### 2. Description of Related Art

In general, surgical removal of tumors has been performed based on a physical removal method and a non-invasive chemical treatment method using a drug. The physical removal method may include, for example, a radiation-assisted surgery.

In the radiation-assisted surgery, a stereotactic frame may be placed on a patient's brain, a lesion region may be accurately identified using computed tomography (CT), magnetic resonance imaging (MRI), and angiography devices based on a type of tumor, and then a treatment plan may be established. Thereafter, the patient may be located on a radiotherapy device such as Gamma Knife, the stereotactic frame may be fixed, and then the surgery may be conducted by irradiating the identified lesion region. The radiation-assisted surgery may be similar to a radiation therapy that uses radiation to reduce or suppress a tumor growth by strongly irradiating a tumor region only. The radiation surgery is an intensive treatment that provides a large amount of radiation to only a tumor region at one time as burning a paper by collecting sunlight in focus with a magnifying glass. In terms of the radiation surgery, it is important to precisely irradiate only the lesion region for a successful treatment. Thus, it is salient that a procedure of identifying an accurate region to be treated in the brain is salient.

A microwave-assisted tumor surgery may be the most recently recognized and developed method. The microwave-assisted tumor surgery may induce apoptosis through coagulation necrosis of tumors by irradiating the tumors with microwaves and vibrating water molecules of tumor cells to generate frictional heat.

### SUMMARY

An aspect provides technology for intensively outputting a microwave signal to a lesion region to generate heat.

Another aspect also provides technology for controlling a phase, an intensity, and a power of a microwave signal to precisely output the microwave signal to a lesion region.

According to an aspect, there is provided a transceiver including a signal attenuator configured to control an intensity of a microwave signal based on corrected information, a phase shifter configured to control a phase of the microwave signal based on the corrected information, a signal switch configured to control a turning-on/off of the microwave signal based on on/off information, a power amplifier configured to amplify the microwave signal, a detector configured to calculate a difference between the microwave signal and a result of the amplifying, and a self-monitoring controller configured to generate the corrected information based on the difference, phase information, and intensity information, output the corrected information to the signal attenuator and the phase shifter, generate the on/off information, and output the on/off information to the signal switch.

The transceiver may further include a directional coupler configured to control a direction of the microwave signal.

The transceiver may further include a signal divider configured to divide the microwave signal and output the divided microwave signal to the signal attenuator and the phase shifter.

The transceiver may further include a circulator configured to prevent a backflow when the microwave signal is transmitted to an antenna.

According to another aspect, there is also provided a therapy system including signal generator configured to generate a microwave signal, a transceiver configured to control a phase, an intensity, and a direction of the microwave signal to output the microwave signal, and a controller configured to control the signal generator based on design data, generate phase information and intensity information based on the design data, and output the phase information and the intensity information to the transceiver, wherein the transceiver includes a signal attenuator configured to control an intensity of the microwave signal based on corrected information, a phase shifter configured to control the phase of the microwave signal based on the corrected information, a signal switch configured to control a turning-on/off of the microwave signal based on on/off information, a power amplifier configured to amplify the microwave signal, a detector configured to calculate a difference between the microwave signal and a result of the amplifying, and a self-monitoring controller configured to generate the corrected information based on the difference, the phase information, and the intensity information, output the corrected information to the signal attenuator and the phase shifter, generate the on/off information, and output the on/off information to the signal switch.

The controller and the transceiver may be connected using a serial data bus.

The transceiver may further include a directional coupler configured to control the direction of the microwave signal.

The transceiver may further include a signal divider configured to divide the microwave signal and output the divided microwave signal to the signal attenuator and the phase shifter.

The transceiver may further include a circulator configured to prevent a backflow when the microwave signal is transmitted to an antenna.

The transceiver may be a plurality of transceivers, and the therapy system may further include a divider configured to divide the microwave signal to output the divided microwave signal to the plurality of transceivers.

The therapy system may further include a therapy apparatus configured to output the microwave signal to a target body.

The therapy apparatus may include a structure provided in a cylindrical shape to encompass the target body, a posture brace configured to set a position and a direction of the target body, an antenna configured to output the microwave signal as a microwave, a cooling liquid configured to assist a transmission of the microwave, and a pressing band configured to prevent a leakage of the cooling liquid.

The antenna may be provided in a form of a 2D horizontal array or a 3D conformal array on a surface of an inner wall of the structure.

A radiating surface of the antenna may be formed on a surface of an inner wall of the structure, a ground surface of the antenna may be formed on a surface of an outer wall of the structure, and the structure may be configured to block an electromagnetic wave.

According to still another aspect, there is also provided a transmission and reception method including controlling an intensity and a phase of a microwave signal based on corrected information, controlling a turning-on/off of the microwave signal based on on/off information, amplifying the microwave signal, calculating a difference between the microwave signal and a result of the amplifying, and generating the corrected information based on the difference, phase information, and intensity information and generating the on/off information

The transmission and reception method may further include controlling a direction of the microwave signal.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 illustrates an example of a target body to be treated according to an example embodiment;

FIG. 2 is a cross-sectional view illustrating an example of the target body of FIG. 1;

FIG. 3 is a block diagram illustrating an example of a therapy system according to an example embodiment;

FIG. 4 is a block diagram illustrating another example of a therapy system according to an example embodiment;

FIG. 5 is a block diagram illustrating an example of a transceiver of FIG. 3;

FIG. 6 is a block diagram illustrating another example of the transceiver of FIG. 3;

FIG. 7A is a front view illustrating an example of the therapy system of FIG. 3;

FIG. 7B is a perspective view illustrating an example of the therapy system of FIG. 3;

FIG. 8A is a cross-sectional view illustrating an example of a target body to which the therapy system of FIG. 3 is applied; and

FIG. 8B is a front view illustrating an example of a target body to which the therapy system of FIG. 3 is applied.

### DETAILED DESCRIPTION

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The following specific structural or functional descriptions are examples to merely describe embodiments, and various alterations and modifications may be made to the examples. Here, the examples are not construed as limited to the disclosure and should be understood to include all changes, equivalents, and replacements within the idea and the technical scope of the disclosure.

Terms such as first, second, A, B, (a), (b), and the like may be used herein to describe components. Each of these terminologies is not used to define an essence, order or sequence of a corresponding component but used merely to distinguish the corresponding component from other component(s). For example, a first component may be referred to a second component, and similarly the second component may also be referred to as the first component.

It should be noted that if it is described in the specification that one component is “connected,” “coupled,” or “joined” to another component, a third component may be “connected,” “coupled,” and “joined” between the first and second components, although the first component may be directly connected, coupled or joined to the second component. In addition, it should be noted that if it is described in the specification that one component is “directly connected” or “directly joined” to another component, a third component may not be present therebetween. Likewise, expressions, for example, “between” and “immediately between” and “adjacent to” and “immediately adjacent to” may also be construed as described in the foregoing.

The terminology used herein is for the purpose of describing particular examples only, and is not to be used to limit the disclosure. As used herein, the terms “a,” “an,” and “the” are intended to include the plural forms as well, unless the context clearly indicates otherwise. As used herein, the terms “include, “comprise,” and “have” specify the presence of stated features, numbers, operations, elements, components, and/or combinations thereof, but do not preclude the presence or addition of one or more other features, numbers, operations, elements, components, and/or combinations thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, reference will now be made in detail to examples with reference to the accompanying drawings, wherein like reference numerals refer to like elements throughout.

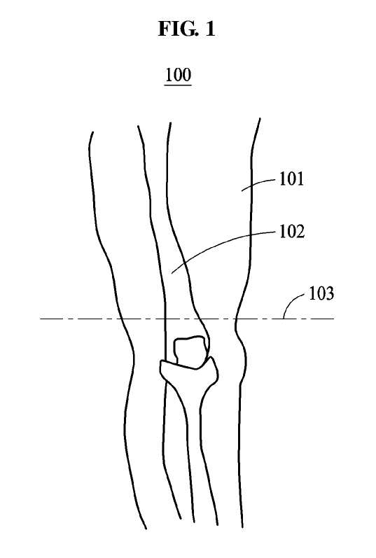

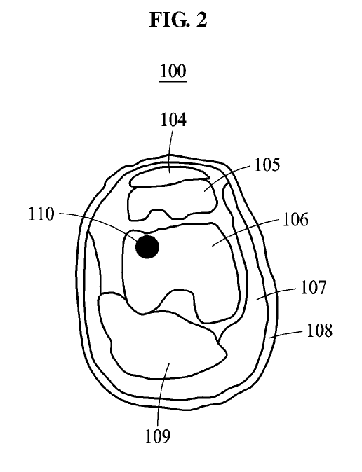

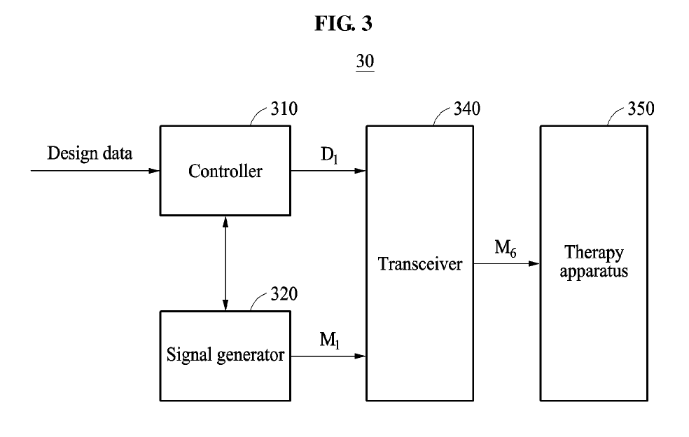

FIG. 1 illustrates an example of a target body to be treated according to an example embodiment, FIG. 2 is a cross-sectional view illustrating an example of the target body of FIG. 1, and FIG. 3 is a block diagram illustrating an example of a therapy system according to an example embodiment.

Referring to FIGS. 1 through 3, a target body 100 may be, for example, a leg 101. The target body 100 may include tissues. For example, the target body 100 may include a bone 102.

FIG. 2 is a cross-sectional view illustrating a cross section 103 of FIG. 1. The target body 100 may include a knee tendon 104, a patella 105, a femur 106, a fat 107, a skin 108, a muscle 109, and a lesion region 110. The lesion region 110 may be a region related to osteosarcoma.

A therapy system 30 may intensively output a microwave to the lesion region 110 for a treatment. The therapy system 30 may be, for example, a topical therapy system.

The therapy system 30 may include a controller 310, a signal generator 320, a transceiver 340, and a therapy apparatus 350.

The controller 310 may receive design data. The design data may include positional information of the lesion region 110 to be treated on the cross section 103 of human tissues, positional information of a tissue, for example, the knee tendon 104, the patella 105, the femur 106, the fat 107, the skin 108, and the muscle 109, and information on a permittivity of the tissue. In this example, the human tissue may be the leg 101. Also, the design data may include information on a turning-on/off of the signal generator 320 or the transceiver 340.

The controller 310 may generate control information D1 based on the design data and transmit the control information D1 to the transceiver 340. The control information D1 may include phase information and intensity information. The phase information and the intensity information may be information on a phase and an intensity of a microwave signal M1.

Also, the controller 310 may control the signal generator 320 or the transceiver 340 based on the design data. The controller 310 may control the signal generator 320 to output the microwave signal M1 or suspend an operation. The controller 310 may control the transceiver 340 to output an amplified microwave signal or suspend an operation. The amplified microwave signal may be a signal of which at least one of a phase, an intensity, and a power is controlled.

The controller 310 and the transceiver 340 may be connected through a data bus. The data bus may be implemented in series or parallel.

The signal generator 320 may generate the microwave signal M1 in response to a control or a request of the controller 310. The microwave signal M1 may be an electromagnetic wave signal having a relatively high frequency. The microwave signal M1 may be, for example, an electromagnetic wave signal having a frequency between 300 megahertz (MHz) and 30 gigahertz (GHz). The microwave signal M1 may have a high frequency and a short wavelength and thus, may be outstanding in straightness, reflection, or absorption performance. The signal generator 320 may transmit the microwave signal M1 to the transceiver 340.

The transceiver 340 may control at least one of the phase and the intensity of the microwave signal M1 based on the control information D1. For example, the transceiver 340 may control at least one of the phase and the intensity of the microwave signal M1 such that the microwave signal M1 is in phase and has a maximum power.

The transceiver 340 may remove an error occurring in a process of controlling the phase, the intensity, or the power of the microwave signal M1. For example, the transceiver 340 may calculate a difference between the microwave signal M1 and the amplified microwave signal and generate an error-free microwave signal M6 based on the difference. The amplified microwave signal may be a signal of which at least one of a phase, an intensity, and a power is controlled. The transceiver 340 may transmit the error-free microwave signal M6 to the therapy apparatus 350.

The therapy apparatus 350 may transmit a microwave that is based on the error-free microwave signal M6, to the lesion region 110. For example, the therapy apparatus 350 may include at least one antenna to transmit the microwave.

The therapy apparatus 350 may be implemented as a cylindrical structure body. Om this example, the at least one antenna may be provided on the cylindrical structure body. The at least one antenna may be provided in a form of a two-dimensional (2D) horizontal array or a three-dimensional (3D) conformal array on a surface of an inner wall of the cylindrical structure body. The at least one antenna may intensively output the microwave to the lesion region 110.

The therapy apparatus 350 may be in direct contact with the target body 100. The therapy apparatus 350 may include a cooling liquid configured to assist a transmission of the microwave. The cooling liquid may include a dielectric material to assist a matching of the microwave. The therapy apparatus 350 may include a pressing band configured to prevent a leakage of the cooling liquid.

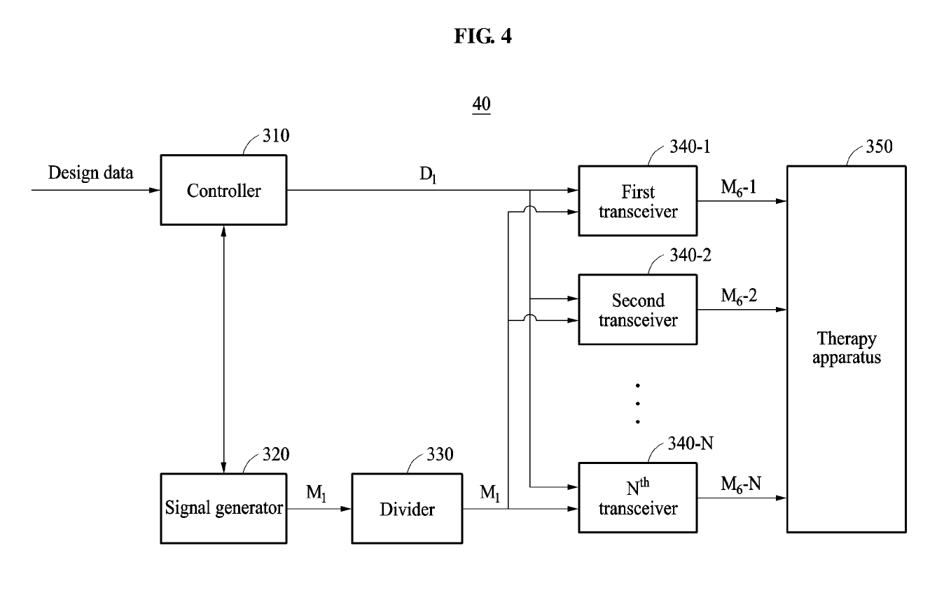

FIG. 4 is a block diagram illustrating another example of a therapy system according to an example embodiment.

Referring to FIG. 4, a therapy system 40 may include the controller 310, the signal generator 320, a divider 330, a plurality of transceivers 340-1 through 340-N, and the therapy apparatus 350.

In an example of FIG. 4, operations and configurations of the controller 310, the signal generator 320, the plurality of transceivers 340-1 through 340-N, and the therapy apparatus 350 may be substantially the same as the operations and configurations of the controller 310, the signal generator 320, the transceiver 340, and the therapy apparatus 350 as described with reference to FIG. 3. Accordingly, the description of FIG. 3 is also applicable to the controller 310, the signal generator 320, and the plurality of transceivers 340-1 through 340-N of FIG. 4.

The divider 330 may divide the microwave signal M1 to generate a plurality of microwave signals. For example, when the number of the plurality of transceivers 340-1 through 340-N is N, the divider 330 may perform 1: N frequency division on the microwave signal M1 and transfer the divided microwave signals to the plurality of transceivers 340-1 through 340-N.

The plurality of transceivers 340-1 through 340-N may generate an amplified microwave signal based on the microwave signal M1 and the control information D1. In this example, a plurality of amplified microwave signals generated by the plurality of transceivers 340-1 through 340-N may be difference from one another. For example, each of the amplified microwave signals may be different in at least one of a phase, an intensity, and a power.

The plurality of transceivers 340-1 through 340-N may control at least one of a phase and an intensity of the microwave signal M1 based on the control information D1. For example, the plurality of transceivers 340-1 through 340-N may control at least one of the phase and the intensity such that the microwave signal M1 is in phase and has a maximum power.

The plurality of transceivers 340-1 through 340-N may remove an error occurring in a process of controlling the phase, the intensity, or the power of the microwave signal M1. For example, the plurality of transceivers 340-1 through 340-N may calculate a difference between the microwave signal M1 and the amplified microwave signal and generate error-free microwave signals, for example, microwave signals M6-1 through M6-N based on the difference. The amplified microwave signal may be a signal of which at least one of a phase, an intensity, and a power is controlled. The plurality of transceivers 340-1 through 340-N may transmit the microwave signals M6-1 through M6-N to the therapy apparatus 350.

The therapy apparatus 350 may transmit a microwave that is based on transmit the microwave signals M6-1 through M6-N, to the lesion region 110. For example, the therapy apparatus 350 may include a plurality of antennas to transmit the microwave.

Although FIG. 4 illustrates that the divider 330 divides the microwave signal M1, embodiments are not limited thereto. Depending on examples, a plurality of signal generators may also be implemented to generate a plurality of microwave signals and transmit the plurality of microwave signals to the plurality of transceivers 340-1 through 340-N.

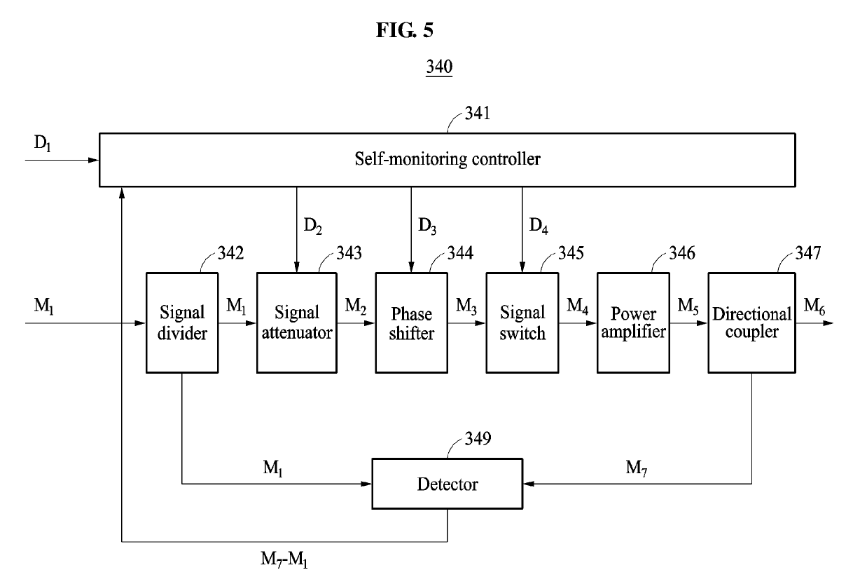

FIG. 5 is a block diagram illustrating an example of a transceiver of FIG. 3.

Referring to FIG. 5, the transceiver 340 may include a self-monitoring controller 341, a signal divider 342, a signal attenuator 343, a phase shifter 344, a signal switch 345, a power amplifier 346, a directional coupler 347, and a detector 349.

The self-monitoring controller 341 may generate corrected information D2 through D4 based on the control information D1. The control information D1 may include phase information and intensity information. The corrected information D2 through D4 may include at least one of intensity-control information, phase-control information, and turning-on/off information.

The self-monitoring controller 341 may transmit the corrected information D2 to the signal attenuator 343. The corrected information D2 may be, for example, the intensity-control information. The signal attenuator 343 may control an intensity of the microwave signal M1 based on the microwave signal M1 and the corrected information D2. The signal attenuator 343 may output an intensity-controlled microwave signal M2 to the phase shifter 344.

The self-monitoring controller 341 may transmit the corrected information D3 to the phase shifter 344. The corrected information D3 may be, for example, the phase-control information. The phase shifter 344 may control a phase of the intensity-controlled microwave signal M2 based on the intensity-controlled microwave signal M2 and the corrected information D3. The phase shifter 344 may output a phase-controlled microwave signal M3 to the signal switch 345.

The self-monitoring controller 341 may transmit the corrected information D4 to the signal switch 345. The corrected information D4 may be, for example, the turning-on/off information. The turning-on/off information may include information used for controlling a turning-on/off of the signal switch 345. The signal switch 345 may output the phase-controlled microwave signal M3 based on the corrected information D4. That is, the self-monitoring controller 341 may determine whether a microwave signal M4 is to be output based on the corrected information D4.

The power amplifier 346 may control a power of the phase-controlled microwave signal M3 to output a microwave signal M5. The power amplifier 346 may be implemented as a high radio frequency (RF) amplifier. For example, the power amplifier 346 may be a last state power amplifier.

The directional coupler 347 may control a direction of the microwave signal M5 to output the microwave signal M6. Also, the directional coupler 347 may perform directional coupling on the partial power of the microwave signal M5, for example, −20 decibels (dB), to output a microwave signal M7 to the detector 349.

The signal divider 342 may divide the microwave signal M1 into two signals. The signal divider 342 may transfer the divided microwave signal M1 to each of the signal attenuator 343 and the detector 349.

The microwave signal M1 may be affected by a disturbance while passing through the signal divider 342, the signal attenuator 343, the phase shifter 344, the signal switch 345, and the power amplifier 346. For example, a difference or an error may occur while each of the signal divider 342, the signal attenuator 343, the phase shifter 344, the signal switch 345, and the power amplifier 346 controls the phase, the intensity, or the power of the microwave signal M1. Hereinafter, an operation of the self-monitoring controller 341 removing the difference or error will be described.

The detector 349 may use the microwave signal M1 as a reference signal reference signal. The detector 349 may calculate a difference (=M1−M7 or M7−M1) between the microwave signal M1 and the microwave signal M7. The detector 349 may transmit the difference between the microwave signal M1 and the microwave signal M7 to the self-monitoring controller 341.

The self-monitoring controller 341 may remove the difference or error based on the difference between the microwave signal M1 and the microwave signal M7. The difference between the microwave signal M1 and the microwave signal M7 may also include a difference or an error in addition to the corrected information D2 through D4.

The self-monitoring controller 341 may remove the difference or error based on the difference between the microwave signal M1 and the microwave signal M7 and the corrected information D2 through D4. For example, the self-monitoring controller 341 may remove the difference or error by subtracting the corrected information D2 through D4 from the difference between the microwave signal M1 and the microwave signal M7.

The self-monitoring controller 341 may update the corrected information D2 through D4 based on the control information D1 and the difference or error. For example, updated information may include a value obtained by subtracting the difference or error from an existing value.

The self-monitoring controller 341 may control the transceiver 340 to output the microwave signal M6 by minimizing the difference or error. Through this, the transceiver 340 may precisely and accurately output the microwave signal M6 to the lesion region 110.

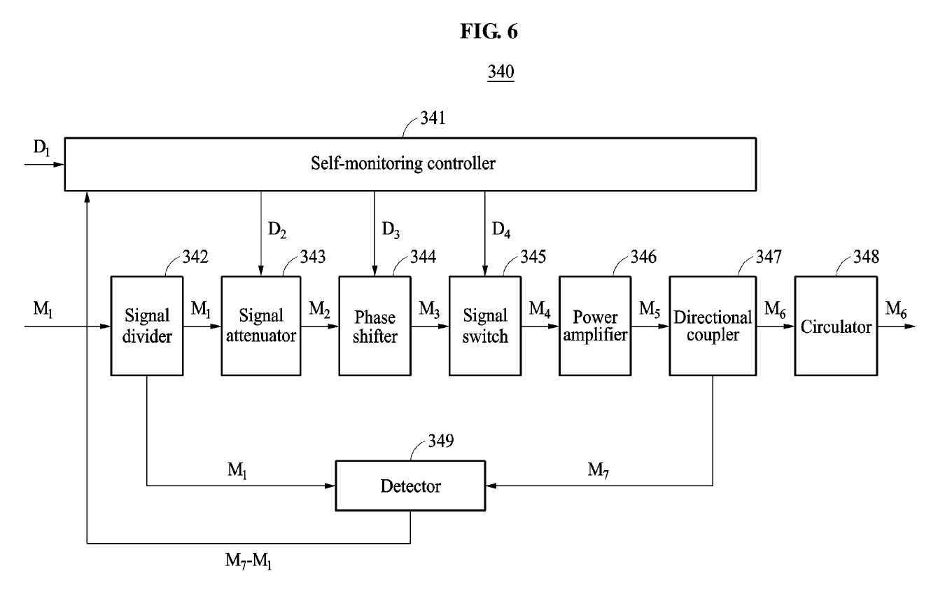

FIG. 6 is a block diagram illustrating another example of the transceiver of FIG. 3.

Referring to FIG. 6, the transceiver 340 may include the self-monitoring controller 341, the signal divider 342, the signal attenuator 343, the phase shifter 344, the signal switch 345, the power amplifier 346, the directional coupler 347, and the detector 349. The transceiver 340 may further include a circulator 348.

In an example of FIG. 6, operations and configurations of the self-monitoring controller 341, the signal divider 342, the signal attenuator 343, the phase shifter 344, the signal switch 345, the power amplifier 346, the directional coupler 347, and the detector 349 may be substantially the same as the operations and the configurations of the self-monitoring controller 341, the signal divider 342, the signal attenuator 343, the phase shifter 344, the signal switch 345, the power amplifier 346, the directional coupler 347, and the detector 349 as described with reference to FIG. 5. For brevity of description, repeated descriptions with respect to the self-monitoring controller 341, the signal divider 342, the signal attenuator 343, the phase shifter 344, the signal switch 345, the power amplifier 346, the directional coupler 347, and the detector 349 will be omitted.

The circulator 348 may prevent a backflow when the error-free microwave signal M6 is transmitted to the therapy apparatus 350. For example, the circulator 348 may prevent the backflow based on a Faraday rotation. The Faraday rotation may indicate a phenomenon that a microwave signal rotates when reaching a magnetic field.

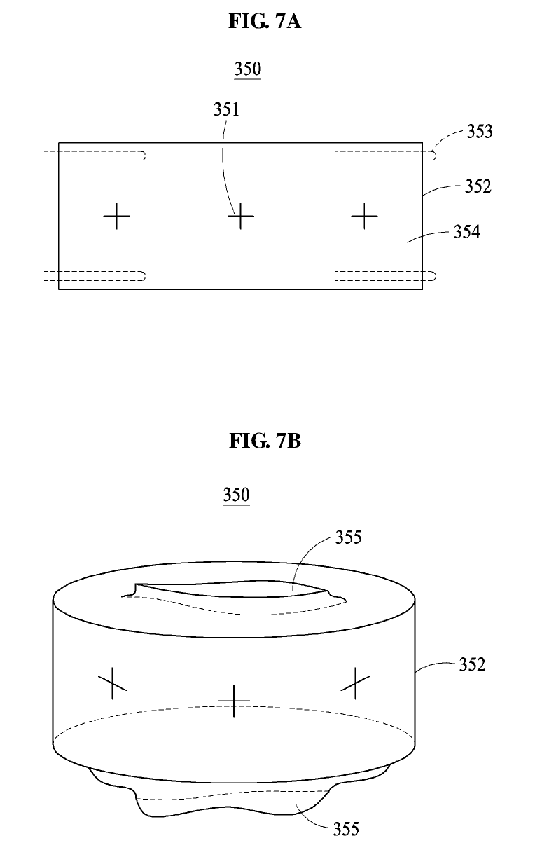

FIG. 7A is a front view illustrating an example of the therapy system of FIG. 3 and FIG. 7B is a perspective view illustrating an example of the therapy system of FIG. 3.

Referring to FIGS. 7A and 7B, the therapy apparatus 350 may include an antenna 351, a structure 352, a posture brace 353, a cooling liquid 354, and a pressing band 355.

The structure 352 may be provided in a cylindrical shape to encompass the target body 100. The structure 352 may form a therapy space as a closed space.

The antenna 351 may output the microwave signal M6 to the target body 100 as a microwave. The antenna 351 may be provided in a form of a 2D horizontal array or a 3D conformal array on a surface of an inner wall of the structure 352. The antenna 351 may circularly provided to the structure 352 to intensively output the microwave to the target body 100.

A radiating surface of the antenna 351 may be formed on a surface of an inner wall of the structure 352. Also, a ground surface of the antenna 351 may be formed on a surface of an outer wall of the structure 352.

The posture brace 353 may set a position and a direction of the target body 100. For example, the posture brace 353 may set a position and a direction of the cross section 103 of the target body 100.

The cooling liquid 354 may assist a transmission of the microwave. The cooling liquid 354 may include a dielectric material to assist a matching of the microwave. For example, the cooling liquid 354 may assist a promotion of the microwave output from the antenna 351 to prevent the microwave from being reflected or refracted on a surface of the target body 100. A permittivity of the cooling liquid 354 may be the same of a permittivity of the target body 100.

The pressing band 355 may prevent a leakage of the cooling liquid 354. The pressing band 355 may be provided in at least one of an upper portion and a lower portion of the structure 352.

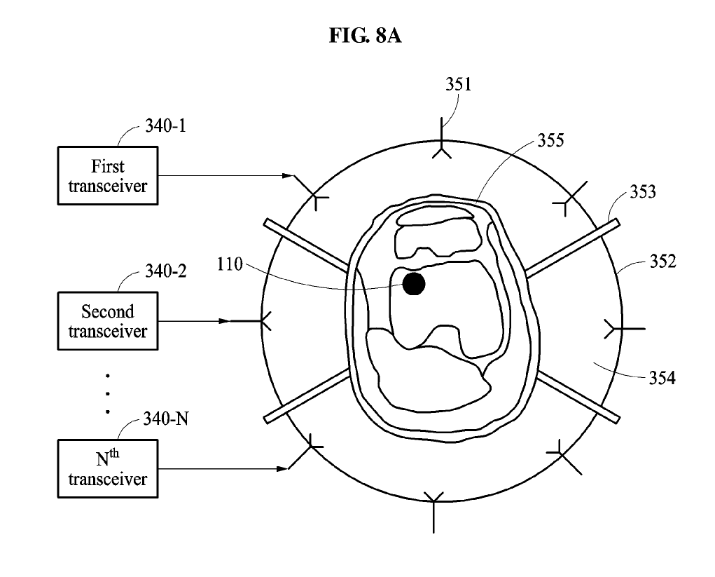

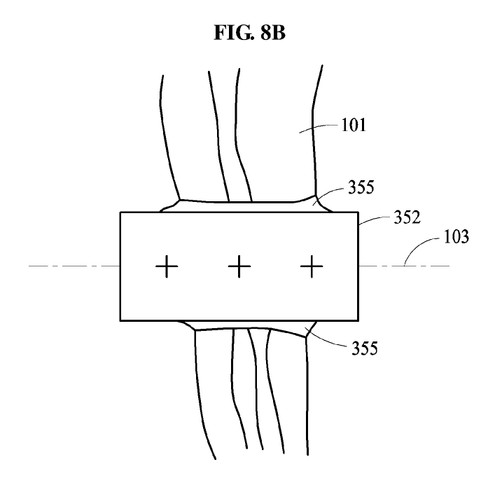

FIG. 8A is a cross-sectional view illustrating an example of a target body to which the therapy system of FIG. 3 is applied and FIG. 8B is a front view illustrating an example of a target body to which the therapy system of FIG. 3 is applied.

Referring to FIGS. 8A and 8B, the therapy apparatus 350 may encompass the target body 100 to form a therapy space as a closed space. A plurality of antennas including the antenna 351 may receive the microwave signals M6-1 through M6-N from the plurality of transceivers 340-1 through 340-N. The plurality of antennas may transmit a microwave to the lesion region 110 based on the microwave signals M6-1 through M6-N. For example, the plurality of antennas may be arranged on the cross section 103 to transmit the microwave in a lateral direction.

Although the foregoing description is provided based on an example in which the target body 100 is the leg 101, embodiments are not limited to the example and thus, the target body 100 may also be a body part, for example, a neck, an arm, an abdomen, and a chest. In this disclosure, a lesion region in a body part may be treated using the microwave.

The components described in the exemplary embodiments of the present invention may be achieved by hardware components including at least one DSP (Digital Signal Processor), a processor, a controller, an ASIC (Application Specific Integrated Circuit), a programmable logic element such as an FPGA (Field Programmable Gate Array), other electronic devices, and combinations thereof. At least some of the functions or the processes described in the exemplary embodiments of the present invention may be achieved by software, and the software may be recorded on a recording medium. The components, the functions, and the processes described in the exemplary embodiments of the present invention may be achieved by a combination of hardware and software.

The processing device described herein may be implemented using hardware components, software components, and/or a combination thereof. For example, the processing device and the component described herein may be implemented using one or more general-purpose or special purpose computers, such as, for example, a processor, a controller and an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and/or multiple types of processing elements. For example, a processing device may include multiple processors or a processor and a controller. In addition, different processing configurations are possible, such as parallel processors.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A transceiver comprising:
a signal attenuator configured to control an intensity of a microwave signal based on corrected information;
a phase shifter configured to control a phase of the microwave signal based on the corrected information;
a signal switch configured to control a turning-on/off of the microwave signal based on on/off information;
a power amplifier configured to amplify the microwave signal;
a detector configured to calculate a difference between the microwave signal and a result of the amplifying; and
a self-monitoring controller configured to generate the corrected information based on the difference, phase information, and intensity information, output the corrected information to the signal attenuator and the phase shifter, generate the on/off information, and output the on/off information to the signal switch.

2. The transceiver of claim 1, further comprising:
a directional coupler configured to control a direction of the microwave signal.

3. The transceiver of claim 1, further comprising:
a signal divider configured to divide the microwave signal and output the divided microwave signal to the signal attenuator and the phase shifter.

4. The transceiver of claim 1, further comprising:
a circulator configured to prevent a backflow when the microwave signal is transmitted to an antenna.

5. A therapy system comprising:
a signal generator configured to generate a microwave signal;
a transceiver configured to control a phase, an intensity, and a direction of the microwave signal to output the microwave signal; and
a controller configured to control the signal generator based on design data, generate phase information and intensity information based on the design data, and output the phase information and the intensity information to the transceiver,
wherein the transceiver includes:
a signal attenuator configured to control an intensity of the microwave signal based on corrected information;
a phase shifter configured to control the phase of the microwave signal based on the corrected information;
a signal switch configured to control a turning-on/off of the microwave signal based on on/off information;
a power amplifier configured to amplify the microwave signal;
a detector configured to calculate a difference between the microwave signal and a result of the amplifying; and
a self-monitoring controller configured to generate the corrected information based on the difference, the phase information, and the intensity information, output the corrected information to the signal attenuator and the phase shifter, generate the on/off information, and output the on/off information to the signal switch.

6. The therapy system of claim 5, wherein the controller and the transceiver are connected using a serial data bus.

7. The therapy system of claim 5, wherein the transceiver further includes:
a directional coupler configured to control the direction of the microwave signal.

8. The therapy system of claim 5, wherein the transceiver further includes:
a signal divider configured to divide the microwave signal and output the divided microwave signal to the signal attenuator and the phase shifter.

9. The therapy system of claim 5, wherein the transceiver further includes:
a circulator configured to prevent a backflow when the microwave signal is transmitted to an antenna.

10. The therapy system of claim 5, wherein the transceiver is a plurality of transceivers, and
the therapy system further includes a divider configured to divide the microwave signal to output the divided microwave signal to the plurality of transceivers.

11. The therapy system of claim 5, further comprising:
a therapy apparatus configured to output the microwave signal to a target body.

12. The therapy system of claim 11, wherein the therapy apparatus includes:
a structure provided in a cylindrical shape to encompass the target body;
a posture brace configured to set a position and a direction of the target body;
an antenna configured to output the microwave signal as a microwave;
a cooling liquid configured to assist a transmission of the microwave; and
a pressing band configured to prevent a leakage of the cooling liquid.

13. The therapy system of claim 12, wherein the antenna is provided in a form of a two-dimensional (2D) horizontal array or a three-dimensional (3D) conformal array on a surface of an inner wall of the structure.

14. The therapy system of claim 12, wherein a radiating surface of the antenna is formed on a surface of an inner wall of the structure,
a ground surface of the antenna is formed on a surface of an outer wall of the structure, and
the structure is configured to block an electromagnetic wave.

15. A transmission and reception method comprising:
controlling an intensity and a phase of a microwave signal based on corrected information;
controlling a turning-on/off of the microwave signal based on on/off information;
amplifying the microwave signal;
calculating a difference between the microwave signal and a result of the amplifying; and
generating the corrected information based on the difference, phase information, and intensity information and generating the on/off information

16. The transmission and reception method of claim 15, further comprising:
controlling a direction of the microwave signal.
